(19) United States
(12) Patent Application Publication
(10) Pub. No.: US 2022/0133121 A1
(43) Pub. Date: May 5, 2022

(54) POLE CLEANING APPARATUS

(71) Applicants: Bryan Nicholas Webb, Kitchener (CA); Elisabetta Sara Gambetti, Kitchener (CA)

(72) Inventors: Bryan Nicholas Webb, Kitchener (CA); Elisabetta Sara Gambetti, Kitchener (CA)

(21) Appl. No.: 17/086,418

(22) Filed: Nov. 1, 2020

(51) Int. Cl. A47L 13/44; A47L 13/17; B08B 9/023; B25G 1/04

(52) U.S. Cl. CPC A47L 13/44; B25G 1/04; B08B 9/023; A47L 13/17

(57) ABSTRACT

A pole cleaning apparatus for cleaning a vertical pole or dance pole is disclosed. The apparatus is configured to enable the user to hygienically and comfortably cleaning the vertical pole or dance pole using a cleaning fabric. The apparatus comprises a handle, an elongated tube having a first end and a second end, and an attachment member. The attachment member is securely affixed to the first end of the elongated tube via threads or using fasteners and/or an adhesive, wherein the attachment member is configured to securely engage a loop or at least a portion of the cleaning fabric, which is securely wrapped around the vertical pole or dance pole using a releasable fabric strip. The handle is slidably affixed to the second end of the elongated tube and configured to enable the user to extend to a length, thereby reaching a great height for hygienically and comfortably cleaning the vertical pole or dance pole using the cleaning fabric with minimal effort.

### BACKGROUND OF THE INNOVATION

### A. Technical Field

The present innovation generally relates to a cleaning apparatus. More specifically, the present innovation relates to a pole cleaning apparatus for simply and hygienically cleaning the contaminated poles, for example, dance poles and vertical poles used in venues such as, but not limited to, fitness centers or gyms, nightclubs, adult entertainment clubs, performance arts venues, dance training centers, and studios, etc.

### B. Description of Related Art

Pole dancing has traditionally formed part of the performance arts. More recently, pole dancing has gained popularity as a form of exercise with increased awareness of the benefits to general strength and fitness. Pole dance combines dance and acrobatics centered around a vertical pole or a dance pole. However, vertical poles or dance poles mostly used in venues such as, but not limited to, fitness centers or gyms, nightclubs, adult entertainment clubs, performance arts venues, dance training centers, and studios simply get contaminated and needs to be cleaned. It is often desirable to clean the vertical poles or dance poles due to accumulated dirt, dust, sweat, and other residues and deposits, etc. Therefore, dance pole cleaning is important to maintain hygiene and it should be done before, during, and after the end of the practice session and/or training and/or before, during, and after performance arts or other dance pieces.

Currently, the vertical poles or dance poles used in venues such as, but not limited to, fitness centers or gyms, nightclubs, adult entertainment clubs, performance arts venues, dance training centers, and studios are cleaned by the people with hands using a wet cloth. However, it is a difficult, tedious, and time-consuming task. Further, it is dangerous to reach the top of the contaminated and dirty vertical pole or dance pole for cleaning. Some prior art devices have been also developed to aid in particular aspects of pole cleaning. However, many of these devices are limited in adjustability, do not allow for great height reach, are not convenient to use, and are not sturdy and durable.

Therefore, there is a need for a pole cleaning apparatus for simply and hygienically cleaning the contaminated poles, for example, dance poles and vertical poles used in venues such as, but not limited to, fitness centers or gyms, nightclubs, adult entertainment clubs, performance arts venues, dance training centers, and studios, etc. Further, there is also a need for a pole cleaning apparatus which is sturdy and durable and also enables the user to adjust for reaching great height, thereby comfortably cleaning the dance pole or vertical pole.

### SUMMARY OF THE INNOVATION

According to an embodiment, a pole cleaning apparatus for simply and safely cleaning the contaminated poles, for example, dance poles and vertical poles used in venues such as, but not limited to, fitness centers or gyms, nightclubs, adult entertainment clubs, performance arts venues, dance training centers, and studios, etc. is disclosed.

In one embodiment, the pole cleaning apparatus is configured to enable the user to hygienically and comfortably cleaning the vertical pole or dance pole using a cleaning fabric with minimal effort. In one embodiment, the pole cleaning apparatus comprises a handle, an elongated tube having a first end and a second end, and an attachment member. In one embodiment, the attachment member is securely affixed to the first end of the elongated tube via, but not limited to, threads or using fasteners and/or an adhesive. The attachment member is configured to securely engage at least a portion of the cleaning fabric by use of, but not limited to, a hook shape or other attachment method/device. The attachment member having a curved or bent portion at an angle for engaging the cleaning fabric. In one embodiment, the attachment member is at least any one of, but not limited to, a hook member, a U-shaped member, and a C-shaped member. In one embodiment, the handle is slidably affixed to the second end of the elongated tube. The handle is configured to enable the user to extend to a length, thereby reaching a great height for hygienically and comfortably cleaning the vertical pole or dance pole using the cleaning fabric with minimal effort. In one embodiment, the handle could be a static member. In one embodiment, the elongated tube further comprises a locking pin for securing the handle to the first end, thereby enabling the user to simply adjust and extend the handle by releasing the locking pin.

In one embodiment, the cleaning fabric is configured to be detachably wrapped around the outer surface of the vertical pole or dance pole using a releasable fabric strip and move longitudinally along the vertical pole or dance pole for cleaning. In one embodiment, the releasable fabric strip is configured to enable the user to securely and detachably wrap the cleaning fabric around the outer surface of the vertical pole or dance pole. In one embodiment, the releasable fabric strip could be sewn on the cleaning fabric. In one embodiment, the releasable fabric strip could be, but not limited to, Velcro® or a hook-and-loop fastener.

In one embodiment, the cleaning fabric comprises a microfiber fabric layer and a khaki fabric adjacent to the microfiber fabric layer. The microfiber fabric layer provides superior cleaning by removing dirt, dust, sweat, and other residues and deposits, etc. accumulated on the vertical pole or dancing pole. In one embodiment, the microfiber fabric layer is sewn to the other side of the khaki fabric. In one embodiment, the cleaning fabric could be impregnated with at least any one of, but not limited to, a disinfectant liquid, a cleaning agent, an alcohol-based liquid, and a detergent liquid. In one embodiment, the cleaning fabric could be washable by hand or using a laundry machine, reusable, and convenient to simply detach from the vertical pole or dance pole by detaching the releasable fabric strip.

In one embodiment, the cleaning fabric further comprises a loop, which is extended from at least any one corner or khaki fabric side of the cleaning fabric, thereby securely holding the cleaning fabric around the outer surface of the vertical pole or dance pole by the attachment member and enabling the user to move the cleaning fabric longitudinally along the vertical pole or dance pole for cleaning.

### BRIEF DESCRIPTION OF DRAWINGS

FIG. 1 shows a perspective view of a pole cleaning apparatus used by a user for cleaning a vertical pole or a dance pole in an embodiment of the present innovation.

FIG. 2 shows a perspective view of a handle and an attachment member of the pole cleaning apparatus in one embodiment of the present innovation.

FIGS. 3A-3B show perspective views of a cleaning fabric in one embodiment of the present innovation.

FIG. 4 shows a perspective view of the pole cleaning apparatus in an extended position in one embodiment of the present innovation.

FIG. 5 shows a perspective view of the pole cleaning apparatus used to clean the vertical pole or dance pole without touching it by the user in one embodiment of the present innovation.

### DETAILED DESCRIPTION OF EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

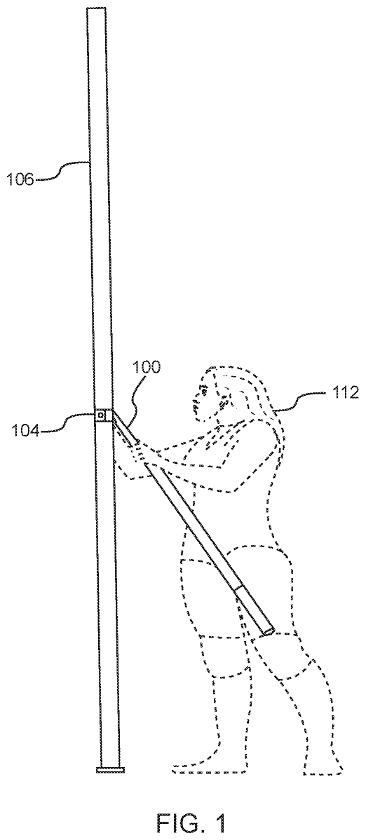

Referring to FIG. 1, a pole cleaning apparatus 100 using by a user 112 for cleaning a vertical pole or a dance pole 106 in one embodiment of the present invention is disclosed. In one embodiment, the pole cleaning apparatus 100 is configured to enable the user 112 to hygienically and comfortably cleaning the vertical pole or dance pole 106 using a cleaning fabric 104 with minimal effort. Mostly, the vertical pole or dance pole 106 used in venues such as, but not limited to, fitness centers or gyms, nightclubs, adult entertainment clubs, performance arts venues, dance training centers, and studios, entertainment industries, etc. gets simply contaminated due to dirt, dust, sweat, and other residues and deposits, etc. so it is often desirable to clean the vertical poles or dance poles 106 before and after use. In one embodiment, the pole cleaning apparatus 100 is further configured to enable the user 112 to adjust to a length for reaching a greater height to hygienically clean the vertical poles or dance poles 106 without touching it.

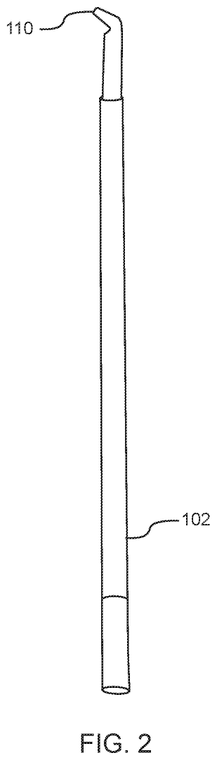

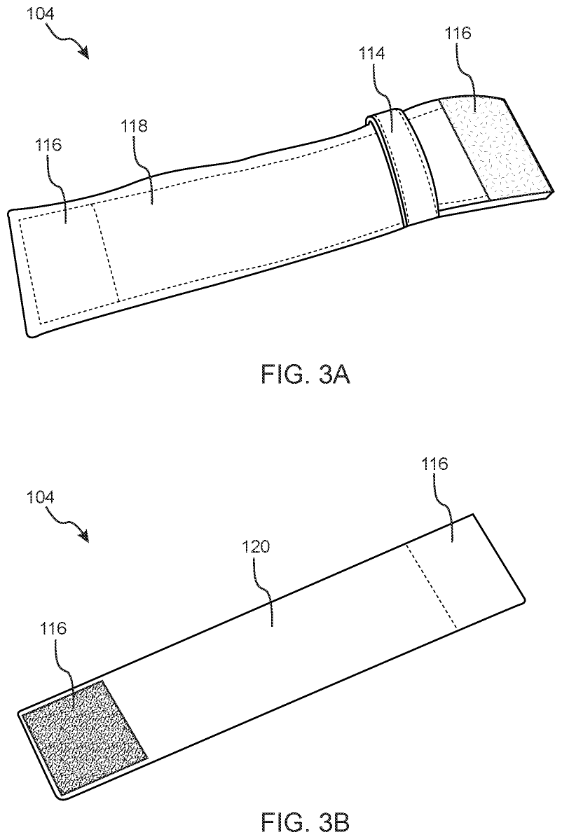

Referring to FIG. 2, a handle 102 and an attachment member 110 of the pole cleaning apparatus 100 in one embodiment of the present invention is disclosed. In one embodiment, the pole cleaning apparatus 100 comprises a handle 102, an elongated tube 108 (shown in FIG. 3) having a first end and a second end, and an attachment member 110. In one embodiment, the attachment member 110 is securely affixed to the first end of the elongated tube 108 via, but not limited to, threads or using fasteners and/or an adhesive. The attachment member 110 is configured to securely engage at least a portion of the cleaning fabric 104 (shown in FIG. 1) by use of, but not limited to, a hook shape or other attachment method/device. The attachment member 110 having a curved or bent portion at an angle for engaging the cleaning fabric 104. In one embodiment, the attachment member 110 is at least any one of, but not limited to, a hook member, a U-shaped member, and a C-shaped member. In one embodiment, the handle 102 is slidably affixed to the second end of the elongated tube 108. The handle 102 is configured to enable the user 112 to extend to a length, thereby reaching a great height for hygienically and comfortably cleaning the vertical pole or dance pole 106 (shown in FIG. 1) using the cleaning fabric 104 with minimal effort. In one embodiment, the handle 102 could be a static member. In one embodiment, the elongated tube 108 further comprises a locking pin for securing the handle 102 to the first end, thereby enabling the user 112 to simply adjust and extend the handle 112 by releasing the locking pin.

Referring to FIGS. 3A-3B, the cleaning fabric 104 of the present invention according to one embodiment is disclosed. In one embodiment, the cleaning fabric 104 is configured to be detachably wrapped around the outer surface of the vertical pole or dance pole 106 using releasable fabric strips 116 and move longitudinally along the vertical pole or dance pole 106 for cleaning. In one embodiment, the releasable fabric strips 116 are configured to enable the user 112 to securely and detachably wrap the cleaning fabric 104 around the outer surface of the vertical pole or dance pole 106. In one embodiment, the releasable fabric strips 116 could be sewn on the cleaning fabric 104. In one embodiment, the releasable fabric strips 116 could be, but not limited to, Velcro® or a hook-and-loop (H&L) fastener.

In one embodiment, the cleaning fabric 104 comprises a microfiber fabric layer 118 and a khaki fabric 120 adjacent to the microfiber fabric layer 118. The microfiber fabric 118 layer provides superior cleaning by removing dirt, dust, sweat, and other residues and deposits, etc. accumulated on the vertical pole or dancing pole 106. In one embodiment, the microfiber fabric layer 118 is sewn to the other side of the khaki fabric 120. In one embodiment, the cleaning fabric 104 could be impregnated with at least any one of, but not limited to, a disinfectant liquid, a cleaning agent, an alcohol-based liquid, and a detergent liquid. In one embodiment, the cleaning fabric 104 could be washable by hand or using a laundry machine, reusable, and convenient to simply detach from the vertical pole or dance pole 106 by detaching the releasable fabric strips 116.

In one embodiment, the cleaning fabric 104 further comprises a loop 114, which is extended from at least any one corner or khaki fabric 120 side of the cleaning fabric 104, thereby securely holding the cleaning fabric 104 around the outer surface of the vertical pole or dance pole 106 by the attachment member 110 and enabling the user 112 to move the cleaning fabric 104 longitudinally along the vertical pole or dance pole 106 for cleaning. In one embodiment, the loop 114 could be sewn on the khaki fabric 120 and near to either side of the releasable fabric strip 116, for example, Velcro® or a H&L fastener.

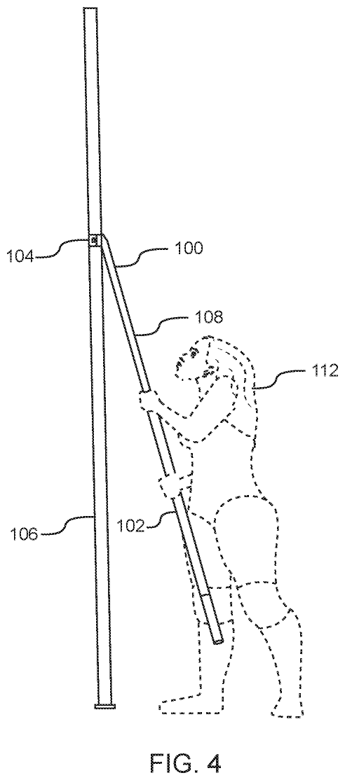

Referring to FIG. 4, the pole cleaning apparatus 100 in an extended position in one embodiment is disclosed. In one embodiment, the pole cleaning apparatus 100 enables the user 112 to adjust and extend the handle 102 for reaching a greater height to clean the vertical pole or dance pole 106. The user 112 could move the cleaning fabric 104 longitudinally along the vertical pole or dance pole 106 for cleaning. In an exemplary embodiment, the elongated tube 108 could be, but not limited to, a broom handle.

In one embodiment, a method for cleaning the vertical pole or dance pole 106 using the pole cleaning apparatus 100 includes multiple steps include, at one step, the cleaning fabric 104 is impregnated with at least any one of, but not limited to, a disinfectant liquid, a cleaning agent or solution, an alcohol-based liquid, and a detergent liquid. At another step, the cleaning fabric 104 is securely wrapped around the vertical pole or dance pole 106 using the releasable fabric strip 116. At another step, the cleaning fabric 104 is moved longitudinally along the vertical pole or dance pole 106 for cleaning using the handle 102 and the attachment member 110, which is securely held and engage the loop 114 of the cleaning fabric 104. Further, the cleaning fabric 104 is detached from the vertical pole or cleaning pole 106. The cleaning fabric 104 could be machine washed using, but not limited to, a laundry machine and reused after washing.

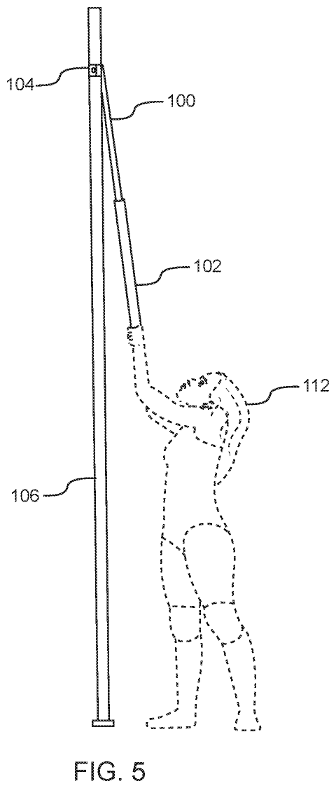

Referring to FIG. 5, the pole cleaning apparatus 100 used to clean the vertical pole or dance pole 106 without touching it by the user 112 in one embodiment is disclosed. In one embodiment, the pole cleaning apparatus 100 could enable the user 112 to hygienically clean the vertical pole or dance pole 106 without touching it. The user 112 could extend the handle 102 to a length, thereby reaching a great height for hygienically and comfortably cleaning the vertical pole or dance pole using the cleaning fabric 104.

Preferred embodiments of this innovation are described herein, including the best mode known to the inventors for carrying out the innovation. It should be understood that the illustrated embodiments are exemplary only and should not be taken as limiting the scope of the innovation.

What is claimed is:

1. A pole cleaning apparatus, comprises:
an elongated tube having a first end and a second end;
an attachment member securely affixed to the first end of the elongated tube, wherein the attachment member is configured to securely engage at least a portion of a cleaning fabric, wherein the attachment member having a curved or bent portion at an angle for engaging the cleaning fabric,
wherein the cleaning fabric is configured to be detachably wrapped around the outer surface of a vertical pole or a dance pole using a releasable fabric strip and move longitudinally along the vertical pole or dance pole for cleaning, and
a handle slidably affixed to the second end of the elongated tube, wherein the handle is configured to enable the user to extend to a length, thereby reaching great height for hygienically and comfortably cleaning the vertical pole or dance pole using the cleaning fabric with minimal effort.

2. The pole cleaning apparatus of claim 1, wherein the handle is configured to extend to a length, thereby enabling the user to reach a top portion of the vertical pole for efficiently cleaning.

3. The pole cleaning apparatus of claim 1, wherein the cleaning fabric comprises a microfiber fabric layer and a khaki fabric adjacent to the microfiber fabric layer, wherein the microfiber fabric layer provides superior cleaning by removing dirt, dust, sweat, and other residues and deposits.

4. The pole cleaning apparatus of claim 1, wherein the cleaning fabric further comprises a loop extends from at least any one corner or khaki fabric side of the cleaning fabric for securely holding the cleaning fabric around the outer surface of the vertical pole or dance pole by the attachment member, thereby enabling the user to move the cleaning fabric longitudinally along the vertical pole or dance pole for cleaning.

5. The pole cleaning apparatus of claim 1, wherein the cleaning fabric is impregnated with at least any one of a disinfectant liquid, a cleaning agent or solution, an alcohol-based liquid, and a detergent liquid.

6. The pole cleaning apparatus of claim 1, wherein the releasable fabric strip is configured to enable the user to securely and detachably wrap the cleaning fabric around the outer surface of the vertical pole or dance pole.

7. The pole cleaning apparatus of claim 1, wherein the releasable fabric strip is Velcro or a hook-and-loop fastener.

8. The pole cleaning apparatus of claim 1, wherein the releasable fabric strip is sewn on the cleaning fabric.

9. The pole cleaning apparatus of claim 1, wherein the cleaning fabric is washable by hand or using a laundry machine, reusable, and convenient to simply detach from the vertical pole or dance pole by detaching the releasable fabric strip.

10. The pole cleaning apparatus of claim 1, wherein the elongated tube further comprises a locking pin for securing the handle to the first end, thereby enabling the user to simply adjust and extend the handle by releasing the locking pin.

11. The pole cleaning apparatus of claim 1, wherein the attachment member is securely affixed to the first end of the elongated tube via threads or using fasteners and/or an adhesive.

12. The pole cleaning apparatus of claim 1, wherein the attachment member is at least any one of a hook member, a U-shaped member, and a C-shaped member.

13. A pole cleaning apparatus, comprises:
an elongated tube having a first end and a second end;
an attachment member securely affixed to the first end of the elongated tube via threads, wherein the attachment member is configured to securely engage a loop of a cleaning fabric, wherein the attachment member having a curved or bent portion at an angle for engaging the cleaning fabric,
wherein the cleaning fabric is configured to be detachably wrapped around the outer surface of a vertical pole or a dance pole using Velcro and move longitudinally along the vertical pole or dance pole for cleaning, and
a handle slidably affixed to the second end of the elongated tube, wherein the handle is configured to enable the user to extend to a length, thereby reaching great height for hygienically and comfortably cleaning the vertical pole or dance pole using the cleaning fabric with minimal effort.

14. The pole cleaning apparatus of claim 13, wherein the attachment member is at least any one of a hook member, a U-shaped member, and a C-shaped member, wherein the attachment member is securely affixed to the first end of the elongated tube using at least any one of fasteners and/an adhesive.

15. The pole cleaning apparatus of claim 13, wherein the cleaning fabric comprises a microfiber fabric layer and a khaki fabric adjacent to the microfiber fabric layer, wherein the microfiber fabric layer provides superior cleaning by removing dirt, dust, sweat, and other residues and deposits.

16. The pole cleaning apparatus of claim 13, wherein the loop extends from at least any one corner or khaki fabric side of the cleaning fabric for securely holding the cleaning fabric around the outer surface of the vertical pole or dance pole by the attachment member, thereby enabling the user to move the cleaning fabric longitudinally along the vertical pole or dance pole for cleaning.

17. The pole cleaning apparatus of claim 13, wherein the cleaning fabric is washable by hand or using a laundry machine, reusable, and convenient to simply detach from the vertical pole or dance pole by using the Velcro®.

18. The pole cleaning apparatus of claim 13, wherein the cleaning fabric is impregnated with at least any one of a disinfectant liquid, a cleaning agent, an alcohol-based liquid, and a detergent liquid.

19. The pole cleaning apparatus of claim 13, wherein the elongated tube further comprises a locking pin for securing the handle to the first end, thereby enabling the user to simply adjust and extend the handle by releasing the locking pin.

20. The pole cleaning apparatus of claim 13, wherein the handle is configured to slide from the second end to the first end, thereby enabling the user to simply adjust and extend the handle by releasing the locking pin.
